(19) United States
(12) Patent Application Publication
(10) Pub. No.: US 2005/0154473 A1
(43) Pub. Date: Jul. 14, 2005

(54) Prosthetic knee mechanism

(76) Inventor: David Bassett, Vista, CA (US)

Correspondence Address: MURPHEY & MURPHEY, A.P.C., 701 PALOMAR AIRPORT ROAD, SUITE 260, CARLSBAD, CA, 92009, US

(21) Appl. No.: 10/756,934

(22) Filed: Jan. 13, 2004

(51) Int. Cl. A61F 2/62

(52) U.S. Cl. 623/43; 623/39

(57) ABSTRACT

A prosthetic knee mechanism that improves stability and flexion of an individual during use. The prosthetic knee mechanism has a braking mechanism to increase stance phase stability in a polycentric knee. The combination of the braking mechanism with a polycentric knee increase stance stability and reduces frictional forces on the prosthetic knee components. The prosthetic knee mechanism uses a cam mechanism to control the flexion and extension of the knee. The cam mechanism also controls the speed at which the foot and shank swing. The prosthetic knee uses a collapsible posterior linkage which allows the knee to go into a small degree of flexion at heel strike while maintaining overall knee stability.

### TECHNICAL FIELD

The present invention generally relates to a prosthetic limb. More specifically, the present invention relates to a knee mechanism that addresses the stability, flexability and flexion of a user during use of the knee. Specifically, the prosthetic knee mechanism uses a posterior linkage, a cam mechanism, and a braking mechanism that allow the knee to bend and shorten when subject to loading by a user.

### BACKGROUND OF THE INVENTION

It is, of course, generally known that prosthetic knee designers have faced very difficult challenges when designing a limb for a user that may have lost one. Historically, when an individual lost a limb, there was very little that an individual could do to replace that limb with any mechanism that would function in the same or similar capacity of the original limb. Many times, an individual would likely have to resort to a crude manipulation of a material such as a peg or wood structure to take the place of the missing limb. However, these limbs did not create the realistic movement of the human limb such as the legs. Specifically, the one aspect of the human leg that has been so difficult to duplicate is the human knee. The knee supports and facilitates the movement of the human foot when in use. However, even modern knee designers have found it difficult to create a mechanism that will simulate the function of the human knee. The problem exists to create a mechanism that will bend effortlessly when desired by the user yet maintain the stability when it is needed in the absence of the musculature that aids the function in the normal human knee.

The prior art in mechanical knee technology shows a variety of different mechanisms that attempt to solve the stability and functionality issues presented with an artificial knee. U.S. Pat. No. 3,863,274 illustrates a design that has a single axis but fails in its ability to allow unrestricted flexion of the knee which ideally begins to occur while weight is still borne by the knee.

Similarly, U.S. Pat. No. 5,800,566 illustrates a multiple bar linkage that has an advantage over the single axis design. The multiple bar linkage may create a higher stability at the heel strike portion of gait but however may lessen stability at toe off. The mechanism may create a limb shortening during knee flexion that aids in toe clearance when the limb swings. However, this multiple bar linkage may fail in its design because it may be overly stable where if an individual has an unnatural gait knee flexion is never initiated. Another problem with the multiple bar linkage can occur when the geometry causes the stability of the knee mechanism to change greatly with small amounts of flexion. The change in geometry can cause serious stumble recovery issues that may make a user uncomfortable with using the mechanism. The geometry problems are affected by forces caused by inclines and decelerations which may cause stability failure. Designers sometimes use hydraulic cylinders to create stability by using the cylinders damping force at different portions of the gait. The weakness in the system is the ability of the cylinder to sense cues during the gait cycle to change the damping of the cylinder. If a non-typical stride causes the knee to miss a cue, the knee stability will be unknown for the user.

Another issue encountered by the prior art is the creation of a mechanism that may allow the foot and shank of a prosthesis to swing through naturally at different stride rates. The most useful design is hydraulic system that forces fluid through an orifice when the knee is bent. These types of hydraulic systems have a tendency to increase the resistance to motion as the speed increases. A problem faced by the hydraulic system is that they tend to be heavy and/or bulky and do not allow for proper damping at different stages in the gait cycle.

Moreover, another obstacle faced in producing a good prosthetic knee is response at heel strike. Ideally, the knee will flex slightly to allow shock to be absorbed. Moreover, the user's center of gravity would not be made to rise. During this slight flexion, the stability of the knee and the prosthetic limb would ideally be maintained. Subsequently, the knee would then extend with minimal energy loss and maintain stability until the user desires it to initiate knee flexion. However, most prior art prosthetic knee mechanism do not flex at heel strike.

U.S. Pat. No. 5,545,232 describes a mechanism that allows flexion at heel strike. However, this mechanism relies on rotating linkages and compression of an elastomer which causes significant amounts of energy to be dissipated.

A need, therefore, exists for an improved prosthetic knee mechanism that solves the above mentioned problems. More specifically, a need, exists for a improved prosthetic knee mechanism that creates stability when needed yet allows for flexion during heel strike.

### SUMMARY OF THE INVENTION

The present invention provides a prosthetic knee mechanism that improves stability and flexion of a user during use. More specifically, the present invention relates to a prosthetic knee mechanism that has a braking mechanism to increase stance phase stability in a polycentric knee. Additionally, the knee uses a cam mechanism to control the flexion and extension of the knee. Moreover, the prosthetic knee may use a collapsible posterior linkage which allows the knee to go into a small degree of flexion at heel strike while maintaining overall knee stability.

To this end, in an embodiment of the present invention, s prosthetic knee mechanism is provided. The knee mechanism has an upper joint member and a lower joint member. The knee mechanism also has a linking assembly disposed between the upper joint member and the lower joint member. Moreover, the knee mechanism also has a mechanical braking mechanism.

In an embodiment, the knee mechanism further comprises an upper joint member wherein the upper joint member forms a polycentric design.

In an embodiment, the knee mechanism further comprises said mechanical braking mechanism which augments friction on the polycentric design thereby increasing stability during use.

In an embodiment, the knee mechanism further comprises the combination of a polycentric design and the mechanical braking mechanism effects geometry of the knee mechanism and further wherein said combination increases stability during use.

In an embodiment of the present invention, a prosthetic knee mechanism is provided. The knee mechanism has an upper joint member and a lower joint member. The knee mechanism also has a linking assembly disposed between the upper joint member and the lower joint member. The knee mechanism has a cam system to control the swing of the foot and shank.

In an embodiment, the knee mechanism further comprises a shaft contained within the cam system wherein said shaft moves within the cam system during knee flexion.

In an embodiment, the knee mechanism further comprises a rod attached to the cam wherein said rod is attached to a piston.

In an embodiment, the knee mechanism further comprises a piston contained within the cam system wherein the piston causes fluid to be displaced from a first side of said piston to a second side of said piston.

In an embodiment, the knee mechanism further comprises a piston contained within the cam system wherein the piston causes fluid to be displaced from a first side of the piston to a second side of said piston through an orifice and further wherein the fluid resistance to flowing through the orifice causes a damping force.

In an embodiment, the knee mechanism further comprises a fluid within said cam system wherein said fluid may be completely passed through an orifice in the cam system wherein said amount of fluid passed through the orifice to effect the damping, speed and swing of the mechanism during use.

In an embodiment, the knee mechanism further comprises said cam mechanism design that may be altered to create alternative damping amounts and a plurality of speed and swing movements by a user.

In an embodiment, the knee mechanism further comprises said cam system that can create different amounts of damping at different stages of the gait cycle.

In an embodiment of the present invention, a prosthetic knee mechanism is provided. The knee mechanism having an upper joint member and a lower joint member and a linking assembly disposed between the upper joint member and the lower joint member. Further, the knee mechanism has a posterior linkage assembly.

In an embodiment, the knee mechanism further comprises said posterior linkage assembly that is disposed between said upper joint member and said lower joint member.

In an embodiment, the knee mechanism further comprises said posterior linkage member that is shortened during used by an individual.

In an embodiment, the knee mechanism further comprises said posterior linkage member that has elastic deformation characteristics.

In an embodiment, the knee mechanism further comprises said posterior linkage member that is comprised of a high tensile metal.

In an embodiment, the knee mechanism further comprises said posterior linkage member that is comprised of a composite material.

In an embodiment, the knee mechanism further comprises said posterior linkage member that is comprised of a plastic material.

In an embodiment of the present invention, a prosthetic knee mechanism is provided. The knee mechanism has an upper joint member and a lower joint member. The knee mechanism also has a linking assembly disposed between the upper joint member and the lower joint member. Additionally, the knee mechanism has a mechanical braking system. The knee mechanism also has a cam system mechanism to control foot and shank speed and posterior linkage system to control knee flexion at heel strike.

It is, therefore, an advantage of the present invention to provide a prosthetic knee mechanism for use by a individual that may provide stability.

Another advantage of the present invention is to provide a prosthetic knee mechanism that may use a mechanical braking mechanism.

Yet another advantage of the present invention is to provide a prosthetic knee mechanism that may use a mechanical braking mechanism to increase stance phase stability in a polycentric knee.

And, another advantage of the present invention is to provide a prosthetic knee mechanism that may use a mechanical braking mechanism to increase stance phase stability in a polycentric knee wherein the knee mechanism may raise the center of rotation at heel strike of the individual during use.

Yet another advantage of the present invention is to provide a prosthetic knee mechanism that may use a mechanical braking mechanism to increase stance phase stability in a polycentric knee wherein the knee mechanism may increase ease of use in initiating knee flexion at toe off.

A still further advantage of the present invention is to provide a prosthetic knee mechanism that may use a mechanical braking mechanism to increase stance phase stability in a polycentric knee wherein the knee mechanism may allow for effective limb length shortening during the swing phase of an individual's natural walk motion.

Yet another advantage of the present invention is to provide a prosthetic knee mechanism having a combination of mechanical brake and a polycentric mechanism that may provide better optimization for knee geometry.

Moreover, an advantage of the present invention is to provide a prosthetic knee mechanism that may be simple to use.

Yet another advantage of the present invention is to provide a prosthetic knee mechanism that may be simple to manufacture.

A further advantage of the present invention is to provide a prosthetic knee mechanism that may use a cam mechanism to control flexion and extension of the prosthetic knee.

A still further advantage of the present invention is to provide a prosthetic knee mechanism that may use a cam mechanism to control flexion and extension of the prosthetic knee wherein the cam mechanism may allow for optimization of the swing characteristics of an individual.

Yet another advantage of the present invention is to provide an prosthetic knee mechanism that may use a cam mechanism to control flexion and extension of the prosthetic knee wherein the cam mechanism may provide effective disengagement of the shank from the damper.

A still further advantage of the present invention is to provide a prosthetic knee mechanism that may use a cam mechanism to control flexion and extension of the prosthetic knee wherein the cam mechanism may provide effective disengagement of the shank from the damper that may minimize the required stroke in the damper.

Yet another advantage of the present invention is to provide a prosthetic knee mechanism that allows for adjustment of the dampening rate of the mechanism.

Still another advantage of the present invention is to provide a prosthetic knee mechanism that may use a cam system wherein the cam system may have a fluid that effects the speed of foot and shank swing.

Yet another advantage of the present invention is to provide a prosthetic knee mechanism that may use a cam system wherein the cam system may have a fluid that effects the damping force, thereby affecting the speed and swing of the foot and shank.

Another advantage of the present invention is to provide a prosthetic knee mechanism that may use a cam system wherein the cam system may have a cam or a plurality of cams.

Still another advantage of the present invention is to provide a prosthetic knee mechanism that may use a cam system wherein the cam system may have a cam that may be produced in a plurality of different sizes.

Yet another advantage of the present invention is to provide a prosthetic knee mechanism that may use a cam system wherein the cam system may have a cam that may be produced in a plurality of different shapes.

A further advantage of the present invention is to provide a prosthetic knee mechanism that may use a cam system wherein the cam system may have a cam that effects the damping force at different stages during a gait cycle of an individual.

A further advantage of the present invention is to provide a prosthetic knee mechanism that may use a collapsible posterior linkage.

Yet another advantage of the present invention is to provide a prosthetic knee mechanism that may use a collapsible posterior linkage which allows the knee to go into a small degree of flexion at heel strike.

Yet another advantage of the present invention is to provide a prosthetic knee mechanism which may use a collapsible posterior linkage which allows the knee to flex a small degree during heel strike yet maintain overall knee stability.

A still further advantage of the present invention is to provide a prosthetic knee mechanism that may reduce the weight needed to produce a prosthetic knee mechanism.

Still another advantage of the present invention is to provide a prosthetic knee mechanism that may use a posterior linkage that may shorten when subject to loading.

Another advantage of the present invention is to provide a prosthetic knee mechanism that may use a posterior linkage wherein the posterior linkage may be comprised of an elastic means.

Still another advantage of the present invention is to provide a prosthetic knee mechanism that may use a posterior linkage wherein the posterior linkage may be comprised of any material that is subject to shortening and/or bending when subject to loading.

Yet another advantage of the present invention is to provide a prosthetic knee mechanism that may use a posterior linkage wherein the posterior linkage may shorten when subject to loading and further wherein the shortening is caused by an elastic deformation of the material that makes up the posterior linkage.

Another advantage of the present invention is to provide a prosthetic knee mechanism that may use a posterior linkage wherein the posterior linkage may be comprised of any high tensile metal and/or composite.

Moreover, an advantage of the present invention is to provide a prosthetic knee mechanism that may reduce the weight of the mechanism such that the knee mechanism is easier for an individual to use.

Yet another advantage of the present invention is to provide a prosthetic knee mechanism that may increase simplicity and weight reduction by elimination of extra mechanisms.

A still further advantage of the present invention is to provide a prosthetic knee mechanism that may reduce energy losses in the use of the knee mechanism.

A further advantage of the present invention is to provide a prosthetic knee mechanism that may reduce the energy loss in rotating mechanisms in the prosthetic knee.

Yet another advantage of the present invention is to provide a prosthetic knee mechanism that may provide increased stability of the knee during use.

Still another advantage of the present invention is to provide a prosthetic knee mechanism that may provide effortless flexion during use of the prosthetic knee.

Another advantage of the present invention is to provide a prosthetic knee mechanism that provides a polycentric linkage with a friction brake to increase stability and provide proper flexion of the prosthetic knee.

Yet another advantage of the present invention is to provide a prosthetic knee mechanism that provides a combination of a polycentric linkage with a friction brake that provides increased stability, rotation, and geometry of the prosthetic knee.

Another advantage of the present invention is to provide a prosthetic knee mechanism that may have a collapsible posterior linkage that allows the top portion of the prosthetic knee to rotate around the anterior bottom axle.

Still another advantage of the present invention is to provide a prosthetic knee mechanism that may combine a polycentric linkage and brake mechanism with a posterior linkage mechanism.

Yet another advantage of the present invention is to provide a prosthetic knee mechanism that may combine a polycentric linkage and brake mechanism with a cam mechanism that may control the foot and shank swing.

Another advantage of the present invention is to provide a prosthetic knee mechanism that may combine a cam mechanism that may control the foot and shank swing of an individual with a posterior linkage to maximize knee flexion at heel strike.

Still another advantage of the present invention is to provide a prosthetic knee mechanism that may combine a cam mechanism that may control the foot and shank swing of an individual with a posterior linkage to maximize knee flexion at heel strike along with a polycentric linkage and brake mechanism to increase stability and allow for proper flexion by an individual.

Additional features and advantages of the present invention are described in, and will be apparent from, the detailed description of the presently preferred embodiments and from the drawings.

### BRIEF DESCRIPTION OF THE DRAWINGS

FIG. 1 illustrates a perspective view of the prosthetic knee mechanism in an embodiment of the present invention.

FIG. 2 illustrates a cross sectional side view of the prosthetic knee mechanism in an embodiment of the present invention.

FIG. 3 illustrates a cross sectional perspective view of the prosthetic knee mechanism in an embodiment of the present invention.

FIG. 4 illustrates a cross sectional perspective view of a section of the prosthetic knee mechanism in an embodiment of the present invention.

FIG. 5 illustrates a top view of the prosthetic knee mechanism in an embodiment of the present invention.

FIG. 6 illustrates a bottom view of the prosthetic knee mechanism in an embodiment of the present invention.

FIG. 7 illustrates a front view of the prosthetic knee mechanism in an embodiment of the present invention.

FIG. 8 illustrates a back view of the prosthetic knee mechanism in an embodiment of the present invention.

### DETAILED DESCRIPTION OF THE PRESENTLY PREFERRED EMBODIMENTS

The present invention relates to a prosthetic knee that improves stability and flexion of a user during use. The mechanism has a braking mechanism to increase stance phase stability in a polycentric knee. Additionally, the knee uses a cam mechanism to control the flexion and extension of the knee. Moreover, the prosthetic knee uses a collapsible posterior linkage which allows the knee a small degree of flexion at heel strike while maintaining overall knee stability.

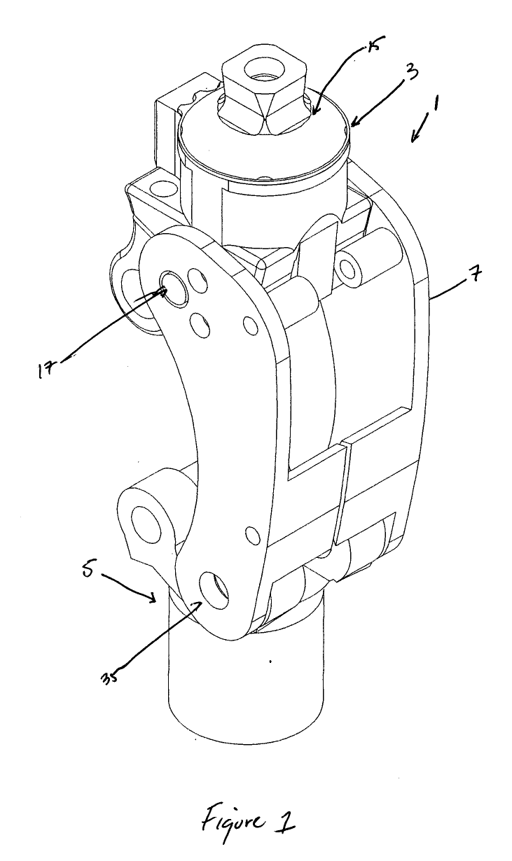

As illustrated in FIG. 1, the present invention generally relates to a prosthetic knee mechanism 1 having an upper portion 3 and a lower portion 5.

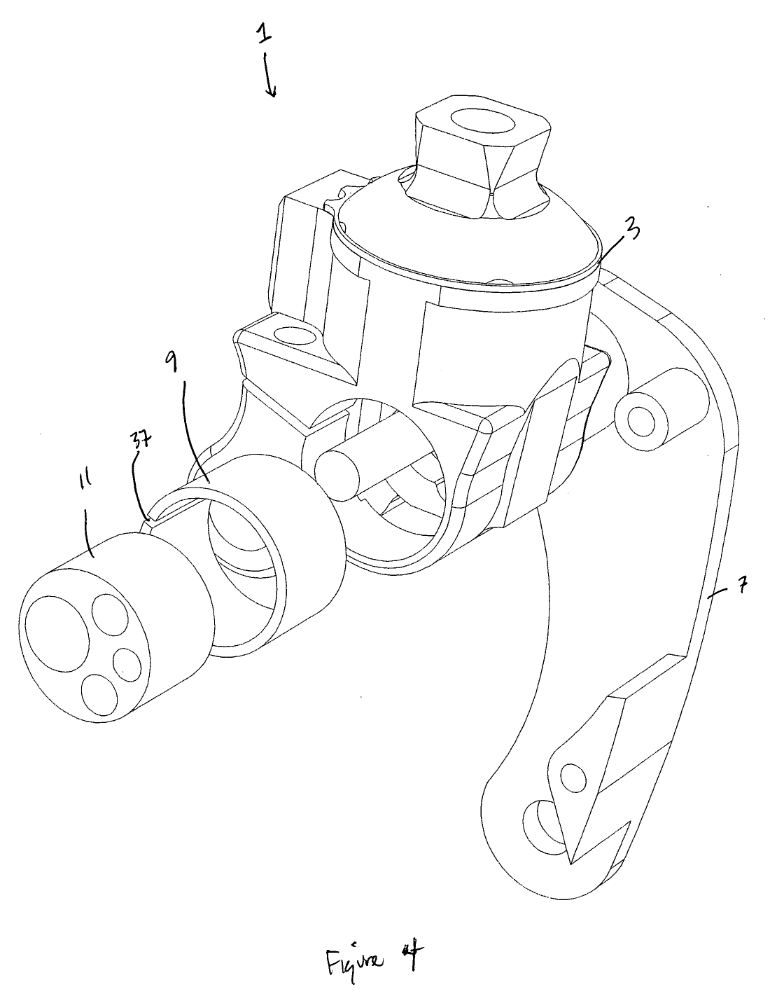

The upper portion 3 may connect to, and/or form part of the thigh component of a prosthesis. The lower portion 5 may connect to, and/or form part of the lower leg system of a prosthesis. A connection means and/or anterior linkage 7 may be positioned between the upper portion 3 and the lower portion 5. The anterior linkage 7 and/or connection means may connect the upper portion 3 to the lower portion 5. The anterior linkage 7 may connect to the upper portion 3 at an upper axis 17, and may connect to the lower portion 5 by a lower axis 35. As illustrated in FIG. 4, the prosthetic knee mechanism 1 may have a braking mechanism 9 that may increase stance phase stability in a polycentric knee 11. The combination of a braking mechanism and a polycentric knee 11 may create stability and proper flexion of the knee during use.

The combination of the braking mechanism 9 and the polycentric knee 11 may create a synergy that provides better stability during heel strike (not shown). At heel strike (not shown) the body of the knee bends and tightens around the knee axle creating a frictional force. A relatively small frictional force is all that is needed to prevent the knee from flexing which will cause less wear of the frictional parts. The augmentation of the braking mechanism 9 on the polycentric knee 11 may allow for the creation of a less stable geometry of the prosthetic knee 1 than other designs. The less stable geometry of the prosthetic knee 1 may ensure that a rapid decrease in stability does not occur on the present invention.

FIG. 4 illustrates the interaction between the braking mechanism 9 and the polycentric knee 11. During heel strike, the braking mechanism may shorten around the polycentric knee 11 thus preventing over extension of the knee 11. The braking mechanism may surround the polycentric knee 11, and may adjust its size in relation to the polycentric knee 11. When braking becomes necessary, the braking mechanism 9 may adjust size to place more friction on the polycentric knee 11 thereby ensuring increased stability at heel strike. Moreover, the braking mechanism 9 may have a space 37 formed therein wherein the space 37 may be closed thereby decreasing the size of the braking mechanism 9 about the polycentric knee 11. However, the braking mechanism may be a configuration that may facilitate the braking and/or increased stability of the polycentric knee 11.

Moreover, the less stable geometry may also assure proper flexion of the prosthetic knee mechanism 1. The polycentric knee 11 may increase the raised instantaneous center of rotation at heel strike. Moreover, the polycentric knee 11 may also increase the ease of initiating knee flexion at toe off and effective limb shortening during swing phase. Furthermore, the combination of the polycentric knee 11 and the braking mechanism 9 may optimize the knee geometry which may ultimately improve gait.

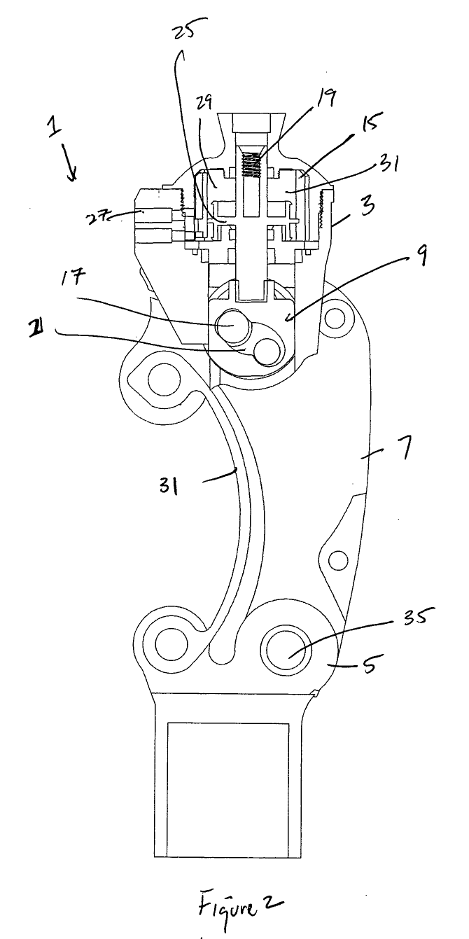

FIG. 1 and 2 illustrate a prosthetic knee mechanism 1 that may use a cam mechanism 15 to control the flexion and extension of the knee mechanism 1. The speed at which the foot(not shown) and the shank swing (not shown) may be controlled by a unique cam mechanism 15.

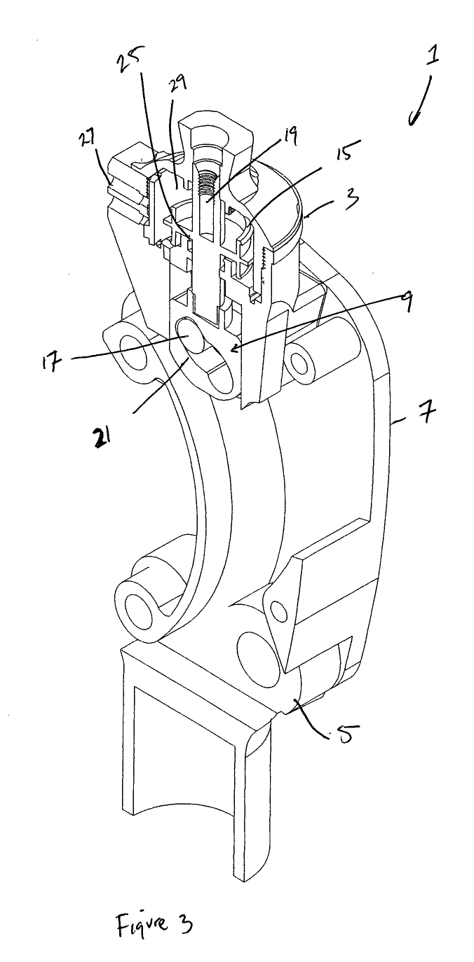

As illustrated in FIG. 2 and FIG. 3, When the knee axle 17 turns during flexion it may cause a shaft 19 to move within a cam 21 which creates a linear progression of the cam 21. The cam 21 is attached to a shank (not shown) which is in turn attached to a piston 25 as illustrated in FIG. 1 and FIG. 2. The movement of the piston 25 may cause fluid to be displaced from one side 29 of the piston 25 to the second side 31 (see FIG. 3). The fluid (not shown) may travel through an orifice 27 when traveling form one side 29 to the second side 31. The fluid (not shown) resistance to flow causes a dampening force which effects the speed of the foot (not shown) and the shank (not shown). The unique cam mechanism 15 may allow the full amount of fluid contained in the system to pass through the orifice 27 with 90 degrees of knee flexion. On extension, the dampening may be restored to previous levels without tampering with the cam system. The use of the cam mechanism 15 may eliminate the need for further damping of the prosthetic knee 1.

Moreover, an advantage of the cam mechanism 15 is the ability to produce the prosthetic knee mechanism 1 in a size that is much smaller, and less bulky than other units. Furthermore, the cam mechanism 15 may allow for alteration of the relative speed of the cam 21 by changing the shape and or size of the cam 21. The cam 21 may be in any form that allows the full amount of fluid (not shown) contained in the system to pass through the orifice 27 with proper knee flexion. The change in cam 21 design may alter the amounts of dampening provided at different stages of the gait cycle of an individual. However, the cam 21 design may be altered to provide effective and efficient dampening needs of the individual user.

FIG. 4 generally illustrates the prosthetic knee mechanism 1 having a polycentric linkage 11 and the braking mechanism 9. FIG. 2 illustrates a collapsible posterior linkage 31 which may allow the knee a small degree of flexion at heel strike. The posterior linkage 31 may bend and effectively shorten when it is subject to weight loading by an individual. During heel strike, the prosthetic knee mechanism 1 may flex by using the posterior linkage 31. The use of a posterior linkage 31 may cause the top portion 3 of the prosthetic knee mechanism 1 to rotate around the anterior bottom axle 35 which in turn may cause the flexion to occur. The shortening and/or bending of the posterior linkage 31 may be caused by an elastic deformation of the material that makes up the posterior linkage 31.

The posterior linkage 31 may be comprised of be any high tensile metal and/or composite. The deformation of the material may exhibit a low amount of hysteresis compared to the compression of an elastomer. This allows a large portion of the energy that causes the deformation to be returned to the form of useful mechanical energy which can be utilized by an individual to cause a progression of the individual's center of gravity.

Moreover, the posterior linkage 31 may increase the simplicity and weight of the prosthetic knee mechanism 1 by eliminating extra mechanisms that may not be needed. Moreover, the posterior linkage 31 may increase the efficiency of the prosthetic knee mechanism 1 by reducing the energy lost in the rotation of other knee mechanisms.

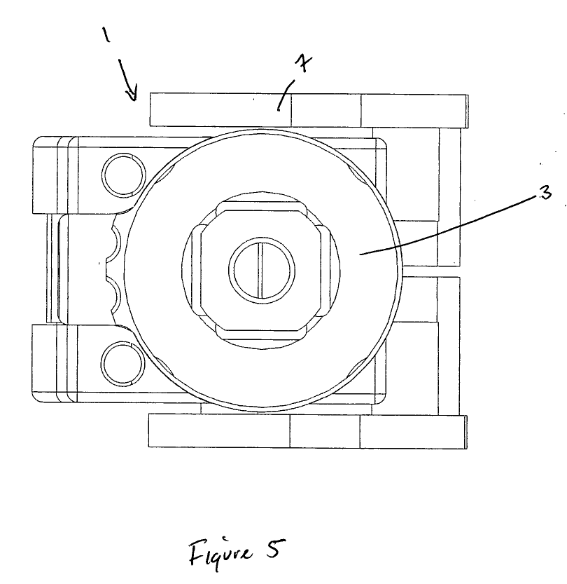

FIG. 5 illustrates the upper portion 3 of the prosthetic knee mechanism 1 that may attach to the limb of a user, and may attach to the lower portion 5 by the connection means and/or anterior linkage 7.

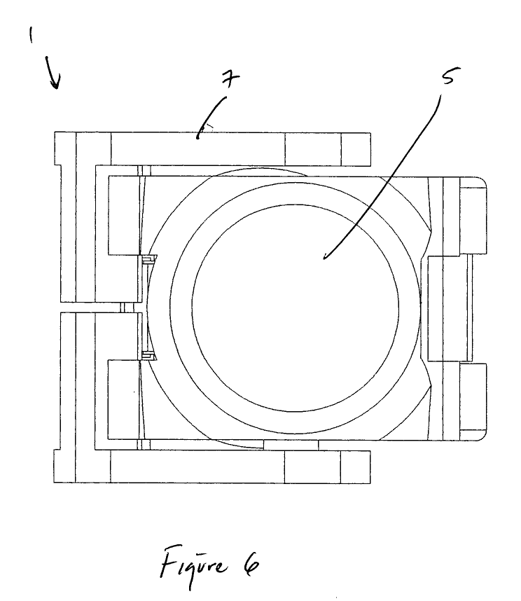

FIG. 6 illustrates the lower portion 5 of the prosthetic knee mechanism 1 that may attach to the shank (not shown) and further may be attached to the upper portion 3 by the connection means and/or anterior linkage 7.

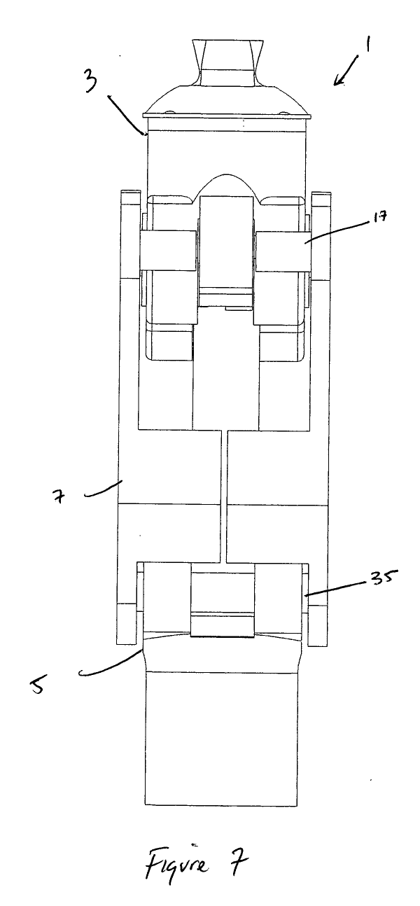

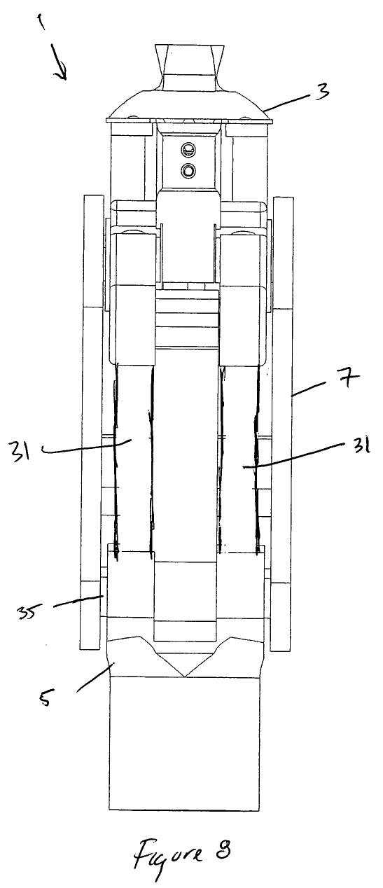

FIGS. 7 and 8 illustrate a front view and a rear view of the prosthetic knee mechanism 1 showing the attachment of the upper portion 3 to the anterior linkage 7 where the anterior linkage 7 may attach to the lower portion 5 by way of a bottom axle 35. The anterior linkage 7 may also be attached to the upper portion 3 by way of a top axle 17. FIG. 8 further illustrates the posterior linkage 31 connected to the top portion 3 and bottom portion 5.

It should be understood that various changes and modifications to the presently preferred embodiments described herein will be apparent to those skilled in the art. Such changes and modifications may be made without departing from the spirit and scope of the present invention and without diminishing its attendant advantages. It is, therefore, intended that such changes and modifications be covered by the appended claims.

1. A prosthetic knee mechanism, the knee comprising:
an upper joint member;
a lower joint member;
a linking assembly disposed between the upper joint member and the lower joint member; and
a mechanical braking mechanism.

2. The knee mechanism of claim 1 further comprising:
an upper joint member wherein the upper joint member forms a polycentric design.

3. The knee mechanism of claim 1 wherein said mechanical braking mechanism augments friction on the polycentric design thereby increasing stability during use.

4. The knee mechanism of claim 1 wherein the combination of a polycentric design and the mechanical braking mechanism effects geometry of the knee mechanism and further wherein said combination increases stability during use.

5. A prosthetic knee mechanism, said knee mechanism comprising:
an upper joint member;
a lower joint member;
a linking assembly disposed between the upper joint member and the lower joint member; and
a cam system to control the swing of the foot and shank.

6. The knee mechanism of claim 5 further comprising:
a shaft contained within the cam system wherein said shaft moves within the cam system during knee flexion.

7. The knee mechanism of claim 5 further comprising:
a shank attached to the cam wherein said shank is attached to a piston.

8. The knee mechanism of claim 5 further comprising:
a piston contained within the cam system wherein the piston causes fluid to be displaced from a first side of said piston to a second side of said piston.

9. The knee mechanism of claim 5 further comprising:
a piston contained within the cam system wherein the piston causes fluid to be displaced from a first side of the piston to a second side of said piston through an orifice and further wherein the fluid resistance to flowing through the orifice causes a damping force.

10. The knee mechanism of claim 5 further comprising:
a fluid within said cam system wherein said fluid may be completely passed through an orifice in the cam system wherein said amount of fluid passed through the orifice to effect the damping, speed and swing of the mechanism during use.

11. The knee mechanism of claim 5 wherein said cam mechanism design may be altered to create alternative damping amounts and a plurality of speed and swing movements by a user.

12. The knee mechanism of claim 5 wherein said cam system can create different amounts of damping at different stages of the gait cycle.

13. A prosthetic knee mechanism, said knee mechanism comprising:
an upper joint member;
a lower joint member;
a linking assembly disposed between the upper joint member and the lower joint member; and
a posterior linkage assembly.

14. The knee mechanism of claim 13 wherein said posterior linkage assembly is disposed between said upper joint member and said lower joint member.

15. The knee mechanism of claim 13 wherein said posterior linkage member is shortened during used by an individual.

16. The knee mechanism of claim 13 wherein said posterior linkage member has elastic deformation characteristics.

17. The knee mechanism of claim 13 wherein said posterior linkage member is comprised of a high tensile metal.

18. The knee mechanism of claim 13 wherein said posterior linkage member is comprised of a composite material.

19. The knee mechanism of claim 13 wherein said posterior linkage member is comprised of a plastic material.

20. A prosthetic knee mechanism, the knee mechanism comprising:
an upper joint member;
a lower joint member;
a linking assembly disposed between the upper joint member and the lower joint member;
a mechanical braking system;
a cam system mechanism to control foot and shank speed; and
a posterior linkage system to control knee flexion at heel strike.

21. The knee mechanism of claim 20 further comprising:
a shaft contained within the cam system wherein said shaft moves within the cam system during knee flexion.

22. The knee mechanism of claim 20 further comprising:
a shank attached to the cam wherein said shank is attached to a piston.

23. The knee mechanism of claim 20 further comprising:
a piston contained within the cam system wherein the piston causes fluid to be displaced from a first side of said piston to a second side of said piston.

24. The knee mechanism of claim 20 further comprising:
a piston contained within the cam system wherein the piston causes fluid to be displaced from a first side of the piston to a second side of said piston through an orifice and further wherein the fluid resistance to flowing through the orifice causes a damping force.

25. The knee mechanism of claim 20 further comprising:
a fluid within said cam system wherein said fluid may be completely passed through an orifice in the cam system wherein said amount of fluid passed through the orifice to effect the damping, speed and swing of the mechanism during use.

26. The knee mechanism of claim 20 wherein said cam mechanism design may be altered to create alternative damping amounts and a plurality of speed and swing movements by a user.

27. The knee mechanism of claim 20 wherein said cam system can create different amounts of damping at different stages of the gait cycle.

28. The knee mechanism of claim 20 wherein said posterior linkage assembly is disposed between said upper joint member and said lower joint member.

29. The knee mechanism of claim 20 wherein said posterior linkage member is shortened during used by an individual.

30. The knee mechanism of claim 20 wherein said posterior linkage member has elastic deformation characteristics.

31. The knee mechanism of claim 20 wherein said posterior linkage member is comprised of a high tensile metal.

32. The knee mechanism of claim 20 wherein said posterior linkage member is comprised of a composite material.

33. The knee mechanism of claim 20 wherein said posterior linkage member is comprised of a plastic material.

34. The knee mechanism of claim 20 further comprising:
an upper joint member wherein the upper joint member forms a polycentric design.

35. The knee mechanism of claim 20 wherein said mechanical braking mechanism augments friction on the polycentric design thereby increasing stability during use.

36. The knee mechanism of claim 20 wherein the combination of a polycentric design and the mechanical braking mechanism effects geometry of the knee mechanism and further wherein said combination increases stability during use.
